(19) United States
(12) Patent Application Publication
(10) Pub. No.: US 2024/0003859 A1
(43) Pub. Date: Jan. 4, 2024

(54) GAS SENSOR

(71) Applicant: Rohm Co., Ltd., Kyoto (JP)

(72) Inventor: Shunsuke AKASAKA, Kyoto (JP)

(21) Appl. No.: 18/345,571

(22) Filed: Jun. 30, 2023

(30) Foreign Application Priority Data
Jul. 4, 2022 (JP) 2022-107603

(51) Int. Cl. G01N 33/00

(52) U.S. Cl. CPC G01N 33/0009

(57) ABSTRACT

A gas sensor includes: a gas detector; and a lid configured to cover the gas detector, wherein the lid is made of a semiconductor material, wherein the lid includes a first main surface facing the gas detector and a second main surface opposite the first main surface, wherein the lid is provided with a plurality of through-holes, each of the plurality of through-holes extending from the first main surface to the second main surface, and wherein each of the plurality of through-holes has a diameter of 50 μm or less.

### CROSS-REFERENCE TO RELATED APPLICATION

This application is based upon and claims the benefit of priority from Japanese Patent Application No. 2022-107603, filed on Jul. 4, 2022, the entire contents of which are incorporated herein by reference.

### TECHNICAL FIELD

The present disclosure relates to a gas sensor.

### BACKGROUND

In the related art, a gas sensor including a gas sensor and an air-permeable cover is disclosed. The air-permeable cover includes a metal lid. The metal lid is provided with a plurality of holes.

### BRIEF DESCRIPTION OF DRAWINGS

The accompanying drawings, which are incorporated in and constitute a part of the specification, illustrate embodiments of the present disclosure.

FIG. 1 is a schematic cross-sectional view of a gas sensor according to a first embodiment of the present disclosure.

FIG. 2 is a schematic plan view of the gas sensor according to the first embodiment.

FIG. 3 is a flowchart of a method of manufacturing the gas sensor according to the first embodiment.

FIG. 4 is a schematic cross-sectional view showing one step of a first example of a method of manufacturing a lid according to the first embodiment.

FIG. 5 is a schematic cross-sectional view showing a step subsequent to the step shown in FIG. 4 in the first example of the method of manufacturing the lid according to the first embodiment.

FIG. 6 is a schematic cross-sectional view showing a step subsequent to the step shown in FIG. 5 in the first example of the method of manufacturing the lid according to the first embodiment.

FIG. 7 is a schematic cross-sectional view showing a step subsequent to the step shown in FIG. 6 in the first example of the method of manufacturing the lid according to the first embodiment.

FIG. 8 is a schematic cross-sectional view showing one step of second and third examples of the method of manufacturing the lid according to the first embodiment.

FIG. 9 is a schematic cross-sectional view showing a step subsequent to the step shown in FIG. 8 in the second and third examples of the method of manufacturing the lid according to the first embodiment.

FIG. 10 is a schematic cross-sectional view showing a step subsequent to the step shown in FIG. 9 in the second example of the method of manufacturing the lid according to the first embodiment.

FIG. 11 is a schematic cross-sectional view showing a step subsequent to the step shown in FIG. 10 in the second example of the method of manufacturing the lid according to the first embodiment.

FIG. 12 is a schematic cross-sectional view showing a step subsequent to the step shown in FIG. 9 in the third example of the method of manufacturing the lid according to the first embodiment.

FIG. 13 is a schematic cross-sectional view showing a step subsequent to the step shown in FIG. 12 in the third example of the method of manufacturing the lid according to the first embodiment.

FIG. 14 is a schematic cross-sectional view of a gas sensor according to a modification of the first embodiment.

FIG. 15 is a schematic cross-sectional view of a gas sensor according to a second embodiment of the present disclosure.

### DETAILED DESCRIPTION

Reference will now be made in detail to various embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, systems, and components have not been described in detail so as not to unnecessarily obscure aspects of the various embodiments.

Details of embodiments of the present disclosure will be described based on the drawings. Throughout the accompanying drawings, the same or corresponding components are denoted by the same reference numerals, and explanation thereof will not be repeated. Configurations of at least parts of the embodiments described below may be combined arbitrarily.

### First Embodiment

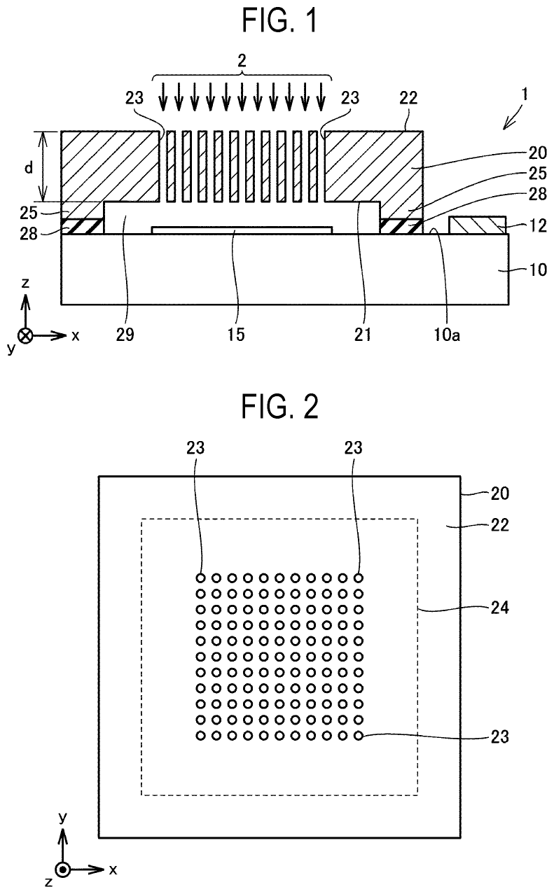

A gas sensor 1 according to a first embodiment of the present disclosure will be described with reference to FIGS. 1 and 2. The gas sensor 1 includes a support substrate 10, a gas detector 15, and a lid 20.

The support substrate 10 is configured to support the gas detector 15. The support substrate 10 is, for example, a printed circuit board. The support substrate 10 includes an electrical wiring (not shown) and an external terminal 12. The support substrate 10 has a main surface 10a. The external terminal 12 is provided, for example, over the main surface 10a. The external terminal 12 is arranged outside the lid 20 in a plan view of the main surface 10a. The external terminal 12 is connected to the electrical wiring of the support substrate 10. The external terminal 12 is connected to the gas detector 15 via the electrical wiring of the support substrate 10.

The gas detector 15 is configured to detect a gas 2 in the surrounding environment of the gas sensor 1. Specifically, the gas detector 15 detects the gas 2 flowed into a cavity 29 of the gas sensor 1 via a plurality of through-holes 23 of the lid 20 and outputs a signal related to the gas 2, such as a concentration of the gas 2. The signal related to the gas 2 is output to the outside of the gas sensor 1 via the electrical wiring and the external terminal 12 of the support substrate 10. The gas 2 detected by the gas detector 15 is not particularly limited, but includes, for example, methane, hydrogen, oxygen, carbon monoxide, nitrogen oxides (NOₓ), or the like. The gas detector 15 is, but not limited to, a semiconductor gas sensor chip, for example.

The lid 20 covers the gas detector 15. The lid 20 is made of a semiconductor material such as silicon (Si). The lid 20 includes a first main surface 21 facing the gas detector 15 and a second main surface 22 opposite the first main surface 21. A distance d between the first main surface 21 and the second main surface 22 is, for example, greater than 0.1 mm. Therefore, a mechanical strength of the lid 20 is improved. The distance between the first main surface 21 and the second main surface 22 may be, for example, 0.5 mm or more, or 1.0 mm or more.

The plurality of through-holes 23 are provided in the lid 20. The plurality of through-holes 23 are in fluid communication with the surrounding environment of the gas sensor 1 and the cavity 29 of the gas sensor 1. Each of the plurality of through-holes 23 extends from the first main surface 21 to the second main surface 22. A diameter of each of the plurality of through-holes 23 is 50 μm or less. Therefore, the lid 20 satisfies IPX4 or higher of a protection grade in a waterproof test (JIS C 0920). The diameter of each of the plurality of through-holes 23 may be 30 μm or less. The diameter of each of the plurality of through-holes 23 is, for example, 20 nm or more. Therefore, a gas permeability of the lid 20 may be prevented from becoming excessively small. The lid 20 may have the protection grade of IPX8 or lower in the waterproof test (JIS C 0920).

An area ratio of the plurality of through-holes 23 is, for example, 10% or more. Therefore, the gas permeability of the lid 20 is improved, and a response speed of the gas sensor 1 is improved. The area ratio of the plurality of through-holes 23 may be 20% or more, or may be 30% or more. The area ratio of the plurality of through-holes 23 is given by dividing an area of the plurality of through-holes 23 in a plan view of the second main surface 22 by an area of a region 24 facing the cavity 29 of the gas sensor 1 among the second main surface 22 in a plan view of the second main surface 22. The gas permeability of the lid 20 is, for example, 100 g/(m²·24 h) or more. The gas permeability of the lid 20 may be 150 g/(m²·24 h) or more, or may be 200 g/(m²·24 h) or more.

The lid 20 is fixed to the support substrate 10 by using a bonding member 28. Specifically, the lid 20 includes a convex portion 25 protruding from the first main surface 21. The convex portion 25 is bonded to the main surface 10a of the support substrate 10 by using the bonding member 28. Therefore, even in a case where the main surface 10a of the support substrate 10 is flat, the cavity 29 in which the gas detector 15 may be accommodated may be formed between the lid 20 and the support substrate 10. The lid 20 and the support substrate 10 form, for example, a chip size package (CSP) for the gas detector 15. The bonding member 28 is, for example, a resin adhesive.

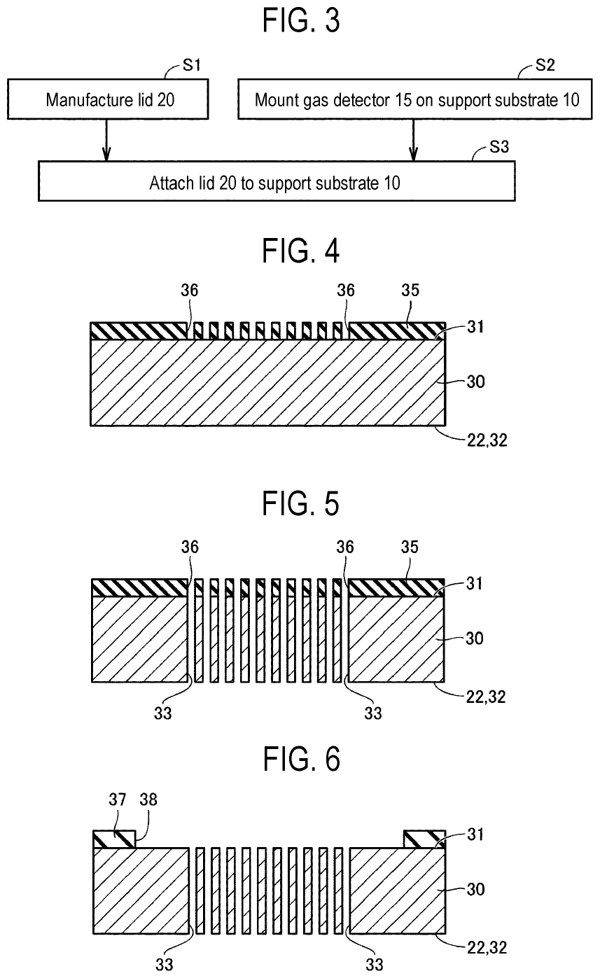

An example of a method of manufacturing the gas sensor 1 according to the present embodiment will be described with reference to FIG. 3.

The lid 20 is manufactured (S1). For example, the lid 20 may be made of a semiconductor material and manufactured by using a semiconductor process. An example of a method of manufacturing the lid 20 will be described later in detail.

The gas detector 15 is mounted over the support substrate 10 (S2). For example, the gas detector 15 is bonded to the support substrate 10 by using a conductive bonding member (not shown) such as solder.

The lid 20 is attached to the support substrate 10 (S3). For example, the lid 20 (specifically, the convex portion 25) is bonded to the main surface 10a of the support substrate by using the bonding member 28. The cavity 29 is formed between the lid 20 and the support substrate 10. The gas detector 15 is accommodated in the cavity 29. The plurality of through-holes 23 of the lid 20 are in fluid communication with the surrounding environment and the cavity 29 of the gas sensor 1.

A first example of a method of manufacturing the lid 20 will be described with reference to FIGS. 4 to 7.

Referring to FIG. 4, a semiconductor substrate 30 is prepared. The semiconductor substrate 30 is, for example, a S1 substrate. The semiconductor substrate 30 includes a main surface 31 and a main surface 32 opposite the main surface 31. The main surface 32 is the second main surface 22 of the lid 20. A resist 35 is formed over the main surface 31. Holes 36 are formed in the resist 35 by patterning the resist 35.

Referring to FIG. 5, through-holes 33 are formed in the semiconductor substrate 30 by etching the semiconductor substrate 30 by using the resist 35 as a mask. The through-holes 33 are formed by deep reactive ion etching (DRIE), for example. The through-holes 33 extending from the main surface 31 to the main surface 32 and having a diameter of 50 μm or less may be easily formed by the DRIE. The resist 35 is removed from the semiconductor substrate 30 by ashing.

Referring to FIG. 6, a resist 37 is formed over the main surface 31. The resist 37 is patterned to form an opening 38 in the resist 37.

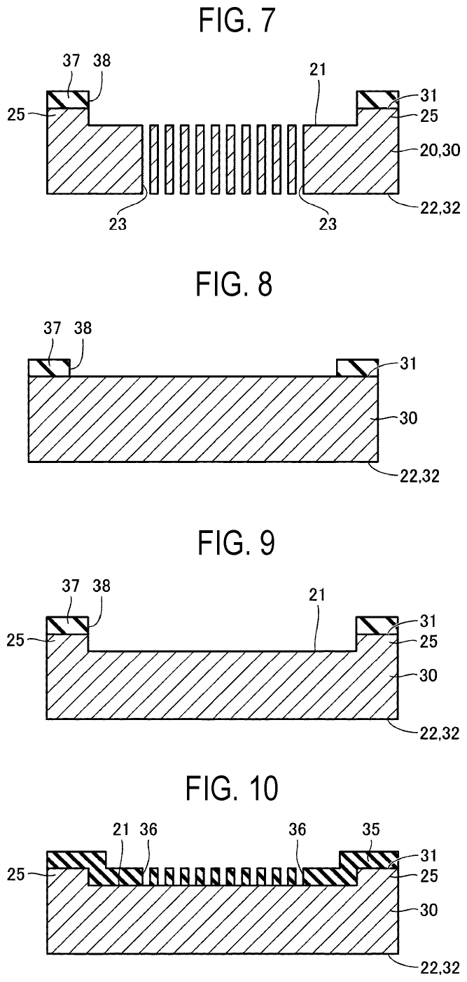

Referring to FIG. 7, the semiconductor substrate 30 is etched by using the resist 37 as a mask to form a concave portion in the main surface 31 of the semiconductor substrate 30. The concave portion is formed by dry etching, for example. A bottom surface of the concave portion is the first main surface 21 of the lid 20. The through-holes 33 become through-holes 23 extending from the first main surface 21 to the second main surface 22. A convex portion 25 is formed in a portion of the semiconductor substrate 30 covered with the resist 37. The resist 37 is removed from the semiconductor substrate 30 by ashing. Thus, the lid 20 is obtained.

A second example of the method of manufacturing the lid 20 will be described with reference to FIGS. 8 to 11. The second example of the method of manufacturing the lid 20 is the same as the first example of the method of manufacturing the lid 20, but in the second example of the method of manufacturing the lid 20, a concave portion is formed in the main surface 31 of the semiconductor substrate 30. Then, through-holes 23 are formed.

Specifically, referring to FIG. 8, a semiconductor substrate 30 is prepared. A resist 37 is formed over the main surface 31 of the semiconductor substrate 30. The resist 37 is patterned to form an opening 38 in the resist 37.

Referring to FIG. 9, the semiconductor substrate 30 is etched by using the resist 37 as a mask to form a concave portion in the main surface 31 of the semiconductor substrate 30. The concave portion is formed by dry etching, for example. The bottom surface of the concave portion is the first main surface 21 of the lid 20. A convex portion 25 is formed in a portion of the semiconductor substrate 30 covered with the resist 37. The resist 37 is removed from the semiconductor substrate 30 by ashing.

Referring to FIG. 10, a resist 35 is formed over the main surface 31 and the first main surface 21. Holes 36 are formed in the resist 35 by patterning the resist 35.

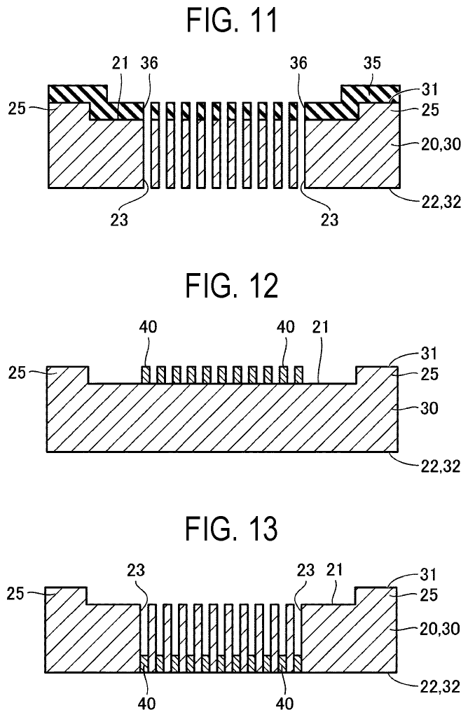

Referring to FIG. 11, the semiconductor substrate 30 is etched by using the resist 35 as a mask to form a plurality of through-holes 23 in the semiconductor substrate 30. The through-holes 23 are formed by deep reactive ion etching (DRIE), for example. The plurality of through-holes 23 extending from the first main surface 21 to the second main surface 22 and having a diameter of 50 μm or less may be easily formed by the DRIE. The resist 35 is removed from the semiconductor substrate 30 by ashing. Thus, the lid 20 is obtained.

A third example of the method of manufacturing the lid 20 will be described with reference to FIGS. 8, 9, 12, and 13. The third example of the method of manufacturing the lid 20 is similar to the second example of the method of manufacturing the lid 20, but is different from the second example of the method of manufacturing the lid 20 in a method of forming the plurality of through-holes 23.

Specifically, as shown in FIGS. 8 and 9, a semiconductor substrate 30 including main surfaces 31 and 32 is prepared, and a concave portion is formed in the main surface 31 of the semiconductor substrate 30. The bottom surface of the concave portion is the first main surface 21 of the lid 20.

As shown in FIGS. 12 and 13, a plurality of through-holes 23 are formed by metal-assisted chemical etching (MaCE).

Specifically, referring to FIG. 12, a dot-shaped metal film 40 is formed over the first main surface 21. The dot-shaped metal film 40 is formed over a region of the first main surface 21 where the plurality of through-holes 23 are formed, but is not formed over a region of the first main surface 21 where the plurality of through-holes 23 are not formed. The dot-shaped metal film 40 is made of, for example, a noble metal such as gold (Au). The dot-shaped metal film 40 is formed by, for example, the following two methods, although not particularly limited.

In a first method of forming the dot-shaped metal film 40, a metal film is formed over the entire first main surface 21, and then the metal film is patterned by etching, lift-off, or the like. The first method of forming the dot-shaped metal film 40 is suitable when the diameter of each of the plurality of through-holes 23 is 0.1 μm or more and 50 μm or less.

In a second method of forming the dot-shaped metal film 40, the dot-shaped metal film 40 is formed over the first main surface 21 by obliquely depositing a metal material over the first main surface 21. By changing a temperature of the semiconductor substrate 30 when depositing the dot-shaped metal film 40, the diameter of each of dot-shaped metal films 40 may be changed. The second method of forming the dot-shaped metal film 40 is suitable when the diameter of each of the plurality of through-holes 23 is 20 nm or more and 0.1 μm or less.

Referring to FIG. 13, the semiconductor substrate 30 is etched by using the dot-shaped metal film 40 as a catalyst. For example, the semiconductor substrate 30 over which the dot-shaped metal film 40 is formed is immersed in an etchant containing an aqueous hydrofluoric acid solution and an oxidizing agent such as a hydrogen peroxide solution. As the etching progresses, the dot-shaped metal film 40 moves in a direction perpendicular to the first main surface 21. The dot-shaped metal film 40 reaches the second main surface 22 to form a plurality of through-holes 23 in the semiconductor substrate 30. The dot-shaped metal film 40 is removed by using an etchant such as an aqua regia-based solution, a cyanogen-based solution, or an iodine-based solution. Thus, the lid 20 is obtained.

The operation and effects of the gas sensor 1 of the present embodiment will be described.

The gas 2 in the surrounding environment of the gas sensor 1 flows into the cavity 29 of the gas sensor 1 via the plurality of through-holes 23 of the lid 20. The gas detector 15 detects the gas 2 which flowed into the cavity 29 and outputs a signal related to the gas 2, such as the concentration of the gas 2. The signal related to the gas 2 is output to the outside of the gas sensor 1 via the electrical wiring (not shown) and the external terminal 12 of the support substrate 10.

The diameter of each of the plurality of through-holes 23 is 50 μm or less. Therefore, the gas 2 may pass through the plurality of through-holes 23, but liquid such as water cannot pass through the plurality of through-holes 23. Therefore, the lid 20 has waterproof performance, and satisfies, for example, IPX4 or higher of the protection grade in the waterproof test (JIS C 0920).

The lid 20 is made of a semiconductor material. The plurality of through-holes 23 may be formed by using a semiconductor process. Therefore, the diameter of each of the plurality of through-holes 23 may be easily reduced to 50 μm or less, and the lid 20 with a waterproof characteristic may be obtained at a low cost. Moreover, since more through-holes 23 may be formed in a narrower region, the area ratio of the plurality of through-holes 23 may be increased. The gas permeability of the lid 20 is improved, and the response speed of the gas sensor 1 is improved.

In contrast, in a gas sensor of a first comparative example, a lid is made of metal. That is, in the lid of the first comparative example, a metal plate is provided with a plurality of through-holes that allow the gas 2 to pass therethrough. However, the diameter of each of the plurality of through-holes that may be formed in the metal plate is 0.1 mm or more. Therefore, the gas sensor of the first comparative example lacks a waterproof characteristic.

Further, in a gas sensor of a second comparative example, a lid is formed of ceramics. That is, in the lid of the second comparative example, a ceramic plate is provided with a plurality of through-holes that allow the gas 2 to pass therethrough. To form the plurality of through-holes with a diameter of 50 μm or less in the ceramic plate, adopting a manufacturing method of erecting a large number of fine pins in a mold cavity, injecting a ceramic base material into the mold cavity, firing the ceramic base material, and pulling out thin pins may be necessary. However, a cost of this manufacturing method is very high. When attempting to manufacture a ceramic lid having a plurality of through-holes at a practical cost, an area density of the plurality of through-holes formed in the lid becomes very small. Therefore, a response speed of the gas sensor of the second comparative example is significantly lowered. Further, in the manufacturing method described above, since long pins may not be erected in the mold, a thickness of the ceramic plate has no choice but to be 0.1 mm or less. Therefore, a mechanical strength of the ceramic lid is low.

### (Modifications)

A gas sensor 1a according to a modification of the present embodiment will be described with reference to FIG. 14.

In the gas sensor 1a, a concave portion is formed in the main surface 10a of the support substrate 10, and the gas detector 15 is mounted over the bottom surface of the concave portion. Specifically, the support substrate 10 includes a main surface 11 recessed from the main surface 10a. The main surface 11 is the bottom surface of the concave portion formed in the main surface 10a. The gas detector 15 is mounted over the main surface 11. The lid 20 does not include the convex portion 25 (see FIG. 1). Since the concave portion is formed in the main surface 10a of the support substrate 10, the cavity 29 in which the gas detector 15 may be accommodated is formed between the lid 20 and the support substrate 10 even without the convex portion 25.

The support substrate 10 includes a pad 13. The pad 13 is provided, for example, over the main surface 11. The pad 13 is connected to the electrical wiring (not shown) of the support substrate 10. The gas detector 15 includes a pad 16. A conductive wire 19, such as an Au wire, is bonded to the pads 13 and 16. The pad 16 is electrically connected to the pad 13 via the conductive wire 19. A signal related to the gas 2, such as the concentration of the gas 2, is output to the outside of the gas sensor 1 via the pad 16, the conductive wire 19, the pad 13, and the electrical wiring and the external terminal 12 of the support substrate 10. For example, the support substrate 10 is a ceramic circuit board, and the lid 20 and the support substrate 10 form a ceramic package for the gas detector 15.

The effects of the gas sensors 1 and 1a of the present embodiment will be described.

The gas sensor 1 and 1a of the present embodiment includes the gas detector 15 and the lid 20 that covers the gas detector 15. The lid 20 is made of a semiconductor material. The lid 20 includes the first main surface 21 facing the gas detector 15, and the second main surface 22 opposite the first main surface 21. The plurality of through-holes 23 are provided in the lid 20. Each of the plurality of through-holes 23 extends from the first main surface 21 to the second main surface 22. The diameter of each of the plurality of through-holes 23 is 50 μm or less.

Since the diameter of each of the plurality of through-holes 23 of the lid 20 is 50 μm or less, the gas sensors 1 and 1a have a waterproof characteristic. Further, the lid 20 is made of a semiconductor material. Therefore, the lid 20 provided with the plurality of through-holes 23 may be obtained at a low cost, such that the cost of the gas sensors 1 and 1a may be reduced.

In the gas sensors 1 and 1a of the present embodiment, the lid 20 satisfies IPX4 or higher of the protection grade in the waterproof test (JIS C 0920). Therefore, the gas sensors 1 and 1a have an improved waterproof characteristic.

In the gas sensors 1 and 1a of the present embodiment, the diameter of each of the plurality of through-holes 23 is 20 nm or more. Therefore, the gas permeability of the lid 20 is prevented from becoming excessively small, and the response speed of the gas sensors 1 and 1a to the gas 2 is prevented from being excessively small.

In the gas sensors 1 and 1a of the present embodiment, the gas permeability of the lid 20 is 100 g/(m²·24 h) or more. Therefore, the response speed of the gas sensors 1 and 1a to the gas 2 may be increased.

The gas sensors 1 and 1a of the present embodiment further include the support substrate 10 configured to support the gas detector 15. The lid 20 is fixed to the support substrate 10. The cavity 29 in which the gas detector 15 is accommodated is formed between the support substrate 10 and the lid 20.

Therefore, the gas detector 15 may be accommodated in the package formed by the lid 20 and the support substrate 10. The package mechanically protects the gas detector 15 and facilitates handling of the gas detector 15.

In the gas sensors 1 and 1a of the present embodiment, the area ratio of the plurality of through-holes 23 is 10% or more. The area ratio of the plurality of through-holes 23 is given by dividing the area of the plurality of through-holes 23 of the second main surface 22 in a plan view by the area of the region 24 facing the cavity 29 among the second main surface 22 of the second main surface 22 in a plan view. Therefore, the response speed of the gas sensors 1 and 1a to the gas 2 may be increased.

In the gas sensor 1 of the present embodiment, the lid 20 includes the convex portion 25 protruding from the first main surface 21. The convex portion 25 is fixed to the support substrate 10.

Therefore, the gas detector 15 may be accommodated in the package formed by the lid 20 and the support substrate 10 even in a case where a concave portion is not formed in the support substrate 10. The package mechanically protects the gas detector 15 and facilitates handling of the gas detector 15.

In the gas sensors 1 and 1a of the present embodiment, the distance d between the first main surface 21 and the second main surface 22 is greater than 0.1 mm. Therefore, the mechanical strength of the lid 20 may be improved.

In the gas sensors 1 and 1a of the present embodiment, the lid 20 is made of S1. Therefore, the cost of the gas sensors 1 and 1a may be reduced.

### Second Embodiment

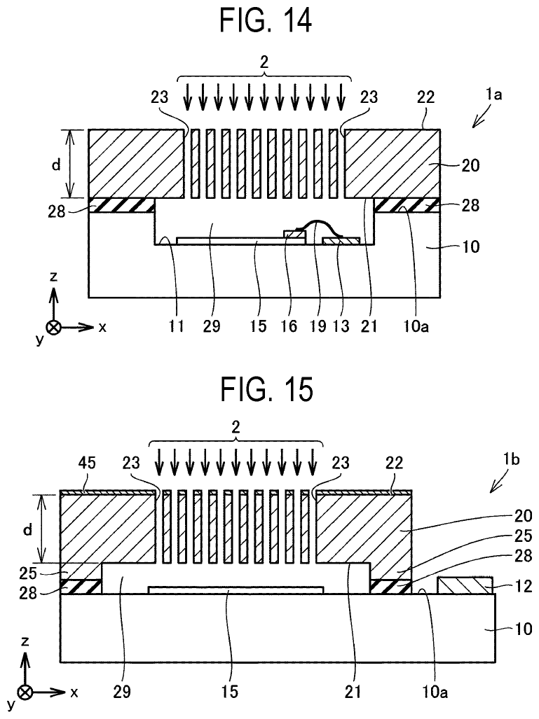

A gas sensor 1b according to a second embodiment of the present disclosure will be described with reference to FIG. 15. The gas sensor 1b of the present embodiment has the same configuration and the same effects as the gas sensor 1 of the first embodiment, but is different from the gas sensor 1 of the first embodiment mainly in the following points.

In the gas sensor 1b, the second main surface 22 of the lid 20 is water-repellent treated. For example, the gas sensor 1b includes a water-repellent layer 45 provided over the second main surface 22 of the lid 20. The water-repellent layer 45 contains a fluorine-based water-repellent agent such as fluorine resin or perfluoroalkyl group-containing silane. Therefore, it is possible to further improve the waterproof characteristic of the gas sensor 1b.

In the modification of the present embodiment, in the gas sensor 1a of the modification of the first embodiment (see FIG. 14), the second main surface 22 of the lid 20 is water-repellent treated.

Hereinafter, various aspects of the present disclosure will be collectively described as Supplementary Notes.

### (Supplementary Notes)

(Supplementary Note 1)
A gas sensor including:
a gas detector; and
a lid configured to cover the gas detector,
wherein the lid is made of a semiconductor material,
wherein the lid includes a first main surface facing the gas detector and a second main surface opposite the first main surface,
wherein the lid is provided with a plurality of through-holes, each of the plurality of through-holes extending from the first main surface to the second main surface, and
wherein each of the plurality of through-holes has a diameter of 50 μm or less.

(Supplementary Note 2)
The gas sensor of Supplementary Note 1, wherein the lid satisfies IPX4 or higher of a protection grade in waterproof test (JIS C 0920).

(Supplementary Note 3)
The gas sensor of Supplementary Note for 2, wherein the diameter is 20 nm or more.

(Supplementary Note 4)
The gas sensor of Supplementary Note 1 or 2, further including a support substrate configured to support the gas detector,
wherein the lid is fixed to the support substrate, and
wherein a cavity in which the gas detector is accommodated is formed between the support substrate and the lid.

(Supplementary Note 5)
The gas sensor of any one of Supplementary Notes 1 to 3, wherein an area ratio of the plurality of through-holes is 10% or more, and
wherein the area ratio is obtained by dividing an area of the plurality of through-holes of the second main surface in a plan view by an area of a region of the second main surface facing the cavity in the plan view.

(Supplementary Note 6)
The gas sensor of Supplementary Note 4 or 5, wherein the lid includes a convex portion that protrudes from the first main surface, and
wherein the convex portion is fixed to the support substrate.

(Supplementary Note 7)
The gas sensor of any one of Supplementary Notes 1 to 6, wherein a gas permeability of the lid is 100 g/(m²·24 h) or more.

(Supplementary Note 8)
The gas sensor of any one of Supplementary Notes 1 to 7, wherein the second main surface of the lid is water-repellent treated.

(Supplementary Note 9)
The gas sensor of any one of Supplementary Notes 1 to 8, wherein a distance between the first main surface and the second main surface is greater than 0.1 mm.

(Supplementary Note 10)
The gas sensor of any one of Supplementary Notes 1 to 9, wherein the lid is made of S1.

It should be considered that the first and second embodiments and their modifications disclosed herein are illustrative in all respects and not restrictive. The scope of the present disclosure is indicated by the scope of the claims rather than the above description, and is intended to include all modifications within the meaning and scope of equivalents of the scope of the claims.

While certain embodiments have been described, these embodiments have been presented by way of example only, and are not intended to limit the scope of the disclosures. Indeed, the embodiments described herein may be embodied in a variety of other forms. Furthermore, various omissions, substitutions and changes in the form of the embodiments described herein may be made without departing from the spirit of the disclosures. The accompanying claims and their equivalents are intended to cover such forms or modifications as would fall within the scope and spirit of the disclosures.

What is claimed is:

1. A gas sensor comprising:
a gas detector; and
a lid configured to cover the gas detector,
wherein the lid is made of a semiconductor material,
wherein the lid includes a first main surface facing the gas detector and a second main surface opposite the first main surface,
wherein the lid is provided with a plurality of through-holes, each of the plurality of through-holes extending from the first main surface to the second main surface, and
wherein each of the plurality of through-holes has a diameter of 50 μm or less.

2. The gas sensor of claim 1, wherein the lid satisfies IPX4 or higher of a protection grade in waterproof test (JIS C 0920).

3. The gas sensor of claim 1, wherein the diameter is 20 nm or more.

4. The gas sensor of claim 1, further comprising a support substrate configured to support the gas detector,
wherein the lid is fixed to the support substrate, and
wherein a cavity in which the gas detector is accommodated is formed between the support substrate and the lid.

5. The gas sensor of claim 4, wherein an area ratio of the plurality of through-holes is 10% or more, and
wherein the area ratio is obtained by dividing an area of the plurality of through-holes of the second main surface in a plan view by an area of a region of the second main surface facing the cavity in the plan view.

6. The gas sensor of claim 4, wherein the lid includes a convex portion that protrudes from the first main surface, and
wherein the convex portion is fixed to the support substrate.

7. The gas sensor of claim 1, wherein a gas permeability of the lid is 100 g/(m²·24 h) or more.

8. The gas sensor of claim 1, wherein the second main surface of the lid is water-repellent treated.

9. The gas sensor of claim 1, wherein a distance between the first main surface and the second main surface is greater than 0.1 mm.

10. The gas sensor of claim 1, wherein the lid is made of S1.
